(19) United States
(12) Patent Application Publication
(10) Pub. No.: US 2014/0257762 A1
(43) Pub. Date: Sep. 11, 2014

(54) METHOD AND DEVICE FOR TRANSFERRING STATICS

(71) Applicant: Gottinger Handelshaus OHG, Zorneding (DE)

(72) Inventor: Johannes PRÖBSTING, Zorneding (DE)

(21) Appl. No.: 14/199,357

(22) Filed: Mar. 6, 2014

(30) Foreign Application Priority Data
Mar. 8, 2013 (DE) 10 2013 102 346.0
Apr. 18, 2013 (DE) 10 2013 103 927.8
May 10, 2013 (DE) 10 2013 104 843.9
Mar. 6, 2014 (DE) 10 2014 102 997.6

(51) Int. Cl. A61F 2/50

(52) U.S. Cl. CPC A61F 2/5046; A61F 2002/505
USPC 703/1

(57) ABSTRACT

Disclosed are a method for transferring statics of an orthopedic test appliance to a definite appliance and a device for performing this method. In accordance with the invention, the inner contour and the outer contour are scanned and aligned relative to each other by means of a protrusion/auxiliary geometry. In a further step, the models resulting from the scanning processes are positioned in a patient-/load-oriented coordinate system.

The invention relates to a method for transferring statics of an orthopedic appliance, for instance, a prosthesis, an orthosis, or a prosthesis shaft, and to a device for performing the method.

In orthopedic technology a problem consists in transferring the geometry of an orthopedic test appliance or of an appliance to be replaced, for instance, a test shaft in the case of providing a lower leg shaft, to the definite shaft. In the following, the term “test appliance” means any orthopedic appliance whose geometry/statics is to be transferred to a definite appliance by means of the method according to the invention.

Presently, the actual provision with a shaft starts with an impression of the amputation stump. Then, a positive model is made of this impression and is modelled in line with particular medical, orthopedic, and biomechanical criteria. Subsequently, a thermoplastic test shaft is produced therefrom, so that the construction of the prosthesis and the fit may be optimized on the patient, and then a definite prosthesis shaft is produced, for instance, with a cast resin method. Particular care has to be taken that the position of an adapter for other prosthesis fit components is transferred from the test shaft to the definite prosthesis shaft as accurately as possible. Conventionally, a transfer device is used into which the grouted test shaft is clamped, wherein the adapter of the test shaft is fixed to the transfer device. After the removal of the test shaft the positive will remain on the transfer device. The definite prosthesis shaft is then manufactured over this positive model, wherein the adapter position is transferred in any plane with sufficient accuracy.

In addition to this largely manual modelling of the test shaft, methods are also known in which the amputation stump is measured by a 3D scanner. Such solutions are, for instance, illustrated in DE 10 2004 007 455 A1, DE 10 2007 014 747 A1, or EP 0 555 207 B1.

EP 1 044 648 B1 discloses a method in which the measuring of an amputation stump is performed by means of reference objects attached thereto, which are then taken from different viewing directions by means of a camera or the like, wherein the contour line of the stump is then determined from the reference distances.

U.S. Pat. No. 6,463,351 B1 describes a method in which a model of the amputation stump is first of all made and this model is then measured via a 3D scanner. Suitable modifications may then be performed on the scan so as to enable an individual adaptation to the patient.

DE 42 32 606 A1 finally illustrates a method in which the inner contour of an orthopedic appliance is scanned and detected by means of a scanner.

It has shown that these known methods do not enable a transfer of statics of an available orthopedic appliance, in the following referred to as test appliance, to an appliance, for instance, a definite shaft, with the required accuracy, or else with extremely high effort only.

Contrary to this it is an object of the invention to provide a method and a device for performing the method by means of which this transfer of statics can be performed with high accuracy and comparatively little effort.

With respect to the method this object is solved with the features of claim 1, and with respect to the device with the features of independent claim 11.

Advantageous further developments of the invention are the subject matter of the subclaims.

The method according to the invention enables the transfer of statics of an available orthopedic test appliance, for instance, a test shaft, to a definite appliance, for instance, a prosthesis shaft. In accordance with the invention, the appliance is first of all grouted with a casting compound pursuant to one variant. After the hardening, this casting compound represents a positive of the inner contour of the appliance. In accordance with the invention, grouting is performed such that a protrusion is formed preferably at the proximal end of the test appliance. This protrusion may also be applied in some other manner.

The protrusion may then be provided with markings, for instance, three markings. Alternatively, it is also possible to form the protrusion itself as a marking contour which enables a later relative positioning of models.

In the following step the test appliance is then scanned along with the protrusion. In this process, the adapter for applying fit components, for instance, a foot of a lower leg prosthesis, should also be scanned if possible.

In a further step the positive is separated from the test appliance and this positive is scanned along with the protrusion.

Alternatively, the inner contour of the test appliance may also be scanned directly, so that grouting is not necessary.

Subsequently, the models of the appliance scan and of the positive/inner contour scan obtained during scanning are stored.

In accordance with the invention, these models are then positioned relatively to each other in that the identical protrusions of the positive/inner contour scan and of the appliance scan are superimposed computationally by means of the markings and/or the marking contour.

After this superimposing, the protrusions that are not required for the actual appliance may be deleted/unmarked, and then the appliance model thus obtained may be stored with its outer and inner contours in a suitable data storage.

This appliance model is then imported to a construction program for generating CAD data of a construction model for the definite orthopedic appliance.

This method enables a transfer of statics with a very low effort, wherein in particular the relative positioning of the two models (test appliance model and positive/inner contour model) is very simple since only the protrusions in accordance with the invention have to be taken into account with this relative positioning.

The processing in the construction program is particularly simple if the appliance model and the positive/inner contour model are available as a point cloud after scanning, wherein these point clouds are then converted to the test appliance model (inner contour and outer contour) by surface reconstruction.

The computational effort during relative positioning may be reduced if, prior to the superimposing of the scans, the non-identical areas are masked, so that only the cutaway models of the test appliance and of the positive and/or the inner contour have to be calculated.

After the superimposing of the cutaway models by means of the marking applied and/or the contour of the protrusion, the point clouds of the actual test appliance and of the positive and/or the inner contour as masked in the afore-explained step are made to overlap with the respectively assigned protrusion, and the point clouds for the test appliance and the positive and/or the inner contour thus aligned are stored separately, but positioned relatively to each other. This means that after this step the models of the test appliance and of the positive/the inner contour are arranged in a common coordinate system.

In accordance with the invention it is preferred if the same coordinate system is assigned to the two models also in the construction program, so that an alignment of the models can be made in this coordinate system. This alignment enables an individual adaptation to the respective patient.

This assignment of the same coordinate system for the arrangement of both models in the space may, for instance, be performed with auxiliary lines assigned during scanning to the test appliance and/or the positive and/or the inner contour.

In one solution, the test appliance model and the positive model/inner contour model are aligned in the construction program in the desired common coordinate system by means of the scanned auxiliary lines.

In an alternative, largely automated solution, this alignment is performed prior to scanning already. This may, for instance, be performed in that the grouted test appliance is aligned in an adjustment device in the desired coordinate system already, wherein the auxiliary lines may serve as orientation during evaluation. Correspondingly, after the removing of the test appliance, the positive remaining in the adjustment device is aligned in this coordinate system and is also scanned. During the importing of the models (appliance and positive) to the construction program, the alignment is then performed automatically in the coordinate system. During the direct scanning of the inner contour this alignment is maintained.

The auxiliary lines may be verticals to a patient's footprint with or without load, dotted function lines of floor reaction forces and of the force behavior with load and without offload on the appliance, or else construction reference lines. In the case of the last described automatic positioning of the models in the predetermined coordinate system it is, on principle, also possible to establish construction planes along the force behavior with load on the appliance.

In one embodiment of the invention the adjustment device is designed with a rotary plate onto which the test appliance is placed. The end section of the test appliance which is remote from the rotary plate is retained in a hinged fastener which is also rotatably retained on the adjustment device. The axes of rotation of the rotary plate and of the fastener are coaxial to each other. A 3D scanner may be held on the adjustment device to be movable in one direction, preferably parallel to this axis of rotation.

The appliance may, for instance, be an orthesis or a prosthesis or a prosthesis shaft.

The device according to the invention for performing the afore-described method comprises a 3D scanner for scanning the outer contour of the test appliance and for scanning the appliance inner contour, wherein a protrusion common in both scans is available.

Furthermore, the device has a data storage for storing the test appliance scan and the positive scan or the inner contour scan. Moreover, there exists an evaluation unit that is designed such that the two models are adapted to be aligned relative to each other by means of their protrusions, and that surface reconstruction of the point cloud models to an appliance model and a positive/inner contour model is enabled from which a construction model is then generated. Correspondingly, the device comprises means for generating CAD data for producing a construction model from the models (test appliance or outer contour model and positive or inner contour model) aligned relative to each other and in a patient-oriented coordinate system.

Such a device enables the transfer of statics from a test appliance to a definite orthopedic appliance with little computational effort, wherein this transfer is performed largely automatically.

This device may additionally be designed with an adjustment device for the defined positioning of the test appliance prior to scanning in a predetermined, preferably patient-oriented coordinate system, wherein load conditions may be taken into account during this positioning.

The evaluation unit mentioned may additionally be adapted to mask the areas of the test appliance point cloud and the positive/inner contour point cloud which do not belong to the protrusion, so that the relative alignment is performed by means of the remaining cutaway models.

The auxiliary lines are particularly simple to apply if the device according to the invention is designed with a light source for imaging auxiliary lines on the test appliance and/or the positive or on the inner contour.

As explained, for determining the inner contour, a scanning process may also be performed for scanning the appliance inner contour instead of producing a positive. In this case, care has to be taken that a marking contour remains on the outer contour and the inner contour which is common to both scans and which simplifies the relative positioning of the scans with respect to each other.

As explained, the markings to be applied may be renounced if the proximal extension is designed with an auxiliary geometry enabling the relative positioning of the models of the appliance outer contour and of the positive as described in the following.

A problem of this proceeding is the digital alignment of the individual scans and/or of the positive and the test appliance with respect to each other. The solution of this problem is the allocation of the same coordinate system for both individual scans. There are two approaches enabling this. The first approach is the distance linking in the construction program itself. This approach is easy to carry out, but has relatively little accuracy. The second approach is a particular proceeding during model digitalization (scan). This proceeding is very accurate, but requires some more effort than the first approach.

The second problem is the alignment of the entire superimposed geometry of outer and inner contour in the space. The solution of this problem is the allocation of a common coordinate system, as it is available on the patient himself/herself. Here, too, there are two approaches. The first approach requires the transfer of auxiliary lines or the like on the test appliance to the construction program. In the second approach, the test appliance is aligned by means of the auxiliary lines in the desired coordinate system already prior to scanning. Then, this desired coordinate system is automatically assigned to the scan and is made a basis in the construction program later on.

In one variant for the allocation of the same coordinate system for the two models, care has to be taken that the markings applied are maintained in the process of the subsequent surface reconstruction. Subsequently, the two models (here outer contour of the test appliance and positive or inner contour) are imported to the construction program and linked with one another by means of the markings. The distances during linking should be kept as small as possible. This constitutes an approach for the allocation of the same coordinate system for both models. This means that the allocation is only performed in the construction program itself.

In the other approach the allocation of the coordinate system is performed in the scan program already. This requires a particular proceeding during the digitalizing of the models which will not be described in detail here. During the importing of the model geometry the same coordinate system in the construction program is assigned to both models (inner/outer contour) and both models are automatically aligned with respect to each other. The result of this approach shows higher accuracy than the afore-described approach.

The second problem is then to process the entire alignment of both models. Here, a coordinate system has to be coordinated in the construction program which, in the optimum case, is equal to the coordinate system defined on the patient. Here, too, there are two approaches enabling a transfer to the construction program. For both approaches it is possible to draw and/or indicate—preferably three—auxiliary lines with load in vertical progression on the appliance. Due to the comparatively simple performance, a front, side and rear view have proven of value in this respect.

In the first approach for solving this problem, care is taken that the three auxiliary lines are visible in the construction program after the surface reconstruction. By means of these markings, three planes positioned vertically on top of each other are then determined. The result thereof is the same alignment of front, side and plan view as is defined and optimized on the patient himself/herself.

In the alternative approach, the test appliance is aligned during scanning already. The test appliance is aligned and/or positioned by means of the vertical markings. Then, the test appliance is separated from the positive and the latter is also scanned, wherein the alignment is maintained.

During the importing to the construction program, the program then performs the alignment of the models as desired and automatically.

If the proceedings are performed correctly, it is possible with the digital appliance construction to adopt and possibly correct, displace or tilt positions of fit components such as prosthesis feet or adapter components, as has proven valuable in the initially described handicraft manufacturing method.

In the afore-described case, in which the test appliance is positioned in the load- or patient-oriented coordinate system, the forming of a protrusion with markings or of a protrusion designed as auxiliary geometry on the positive may on principle also be renounced. This, however, preconditions that the scanner, during the scanning of the inner and outer contours, remains at its predetermined measurement position, so that the scans can then be assumed in the construction program in their position already. The inner contour may be scanned directly, or else a positive may be produced which is then scanned for detecting the inner contour after the removal of the test appliance.

The applicant reserves the right of directing an own independent claim on this proceeding.

Furthermore, the applicant reserves the right of making the adjustment device described the subject matter of an own set of claims.

Preferred embodiments of the invention will be explained in detail in the following by means of schematic drawings. There show:

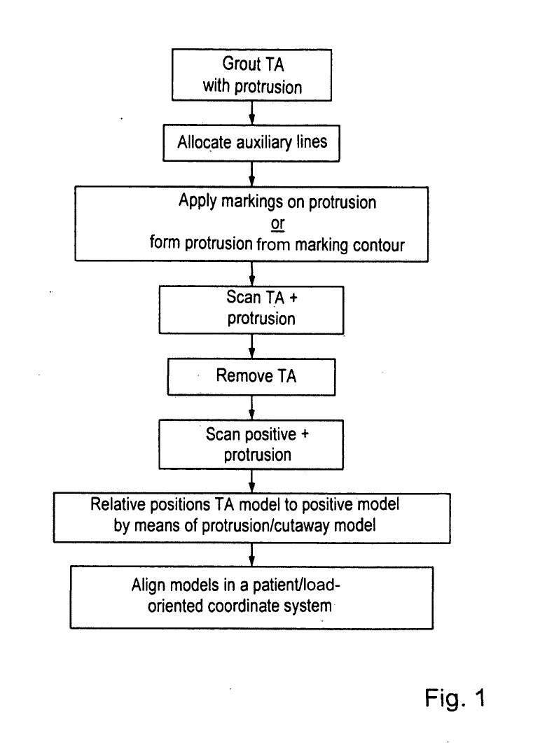

FIG. 1 a strongly simplified flow chart of the method for the transfer of statics according to the invention;

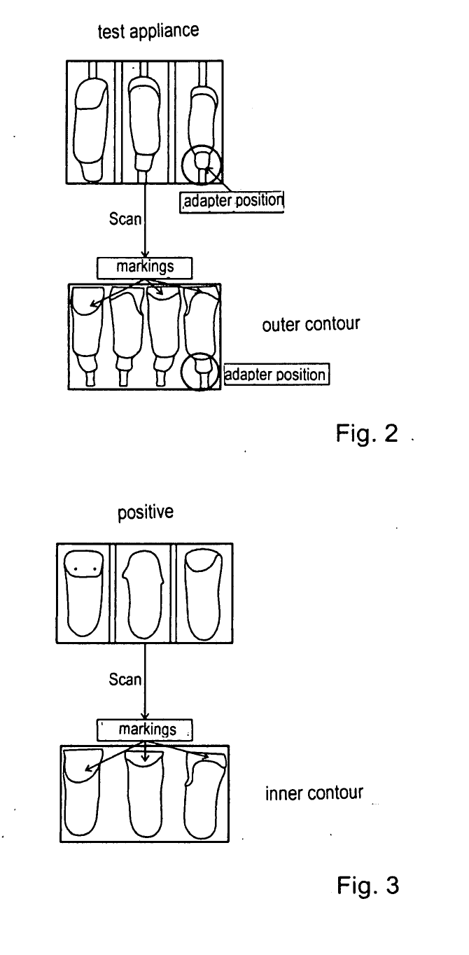

FIG. 2 a scan of an outer contour of a tests appliance, precisely of a test shaft;

FIG. 3 a scan of a positive (inner contour) of the shaft of FIG. 2;

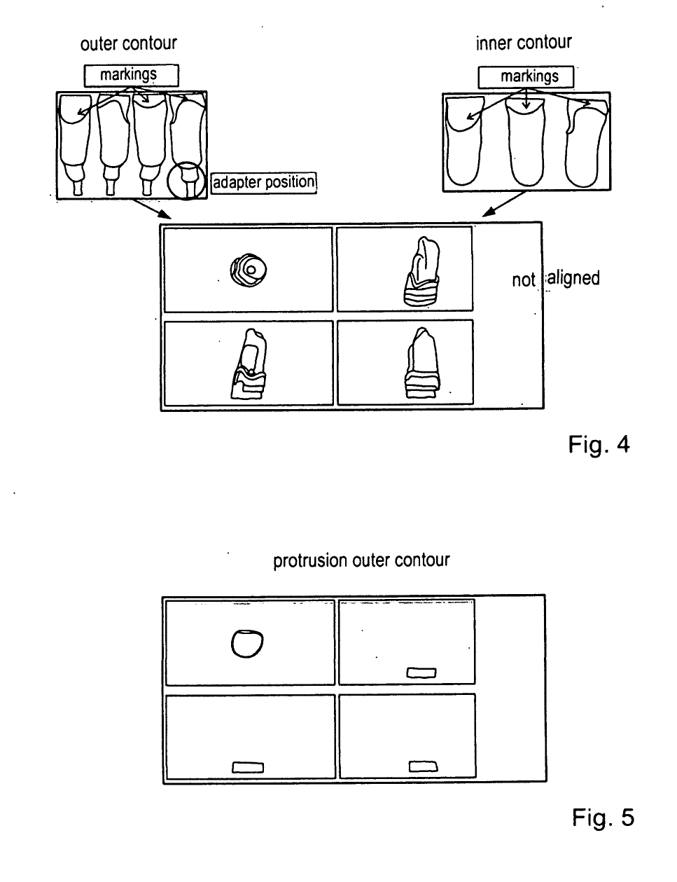

FIG. 4 an illustration of a model generated from the scans pursuant to FIGS. 2 and 3;

FIG. 5 the scan pursuant to FIG. 2, wherein only a protrusion is illustrated;

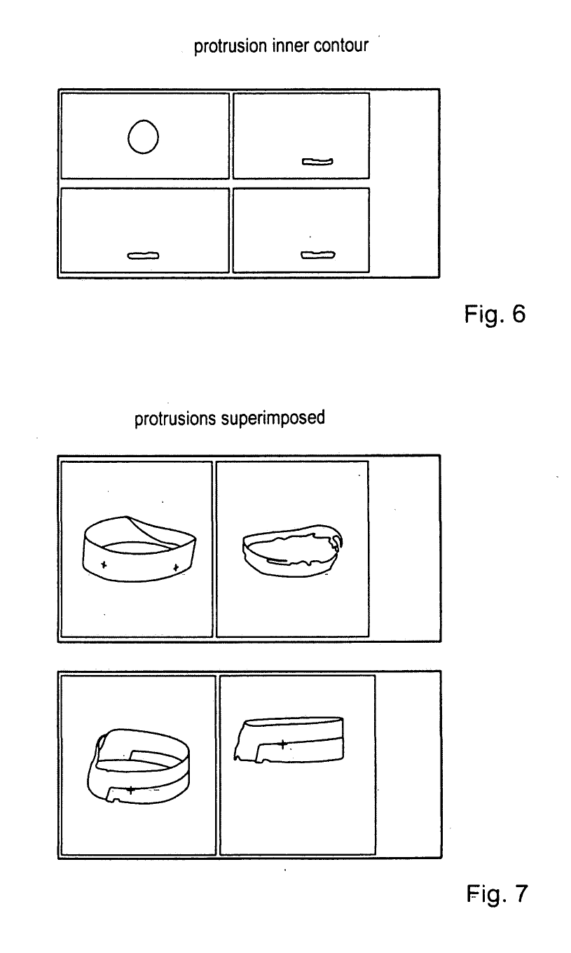

FIG. 6 the scan pursuant to FIG. 3, wherein also only the protrusion is illustrated;

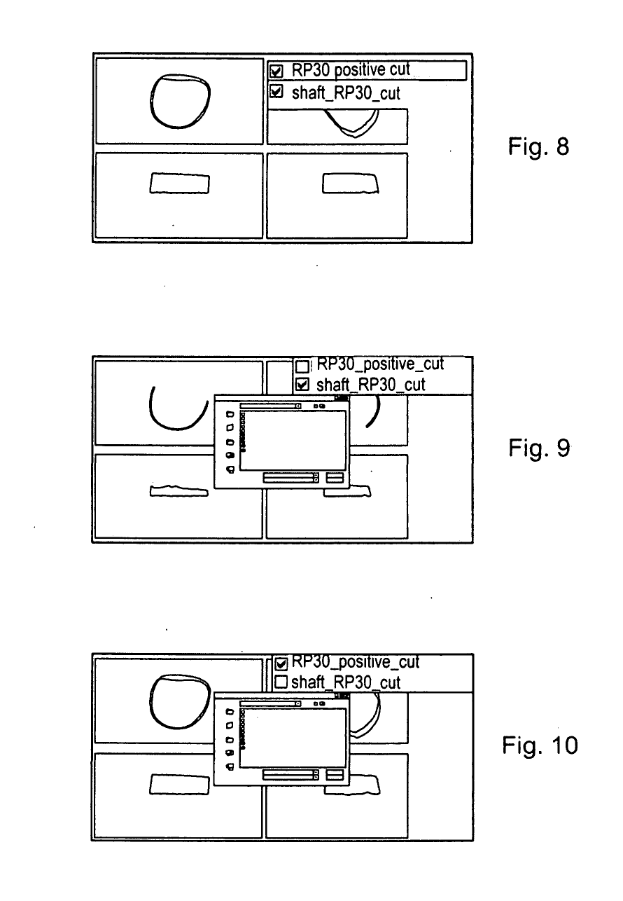

FIGS. 7, 8 the cutaway models pursuant to FIGS. 5 and 6 in the superimposed condition;

FIGS. 9, 10 individual illustrations of the protrusions of the test shaft model and of the positive model;

FIG. 11 the aligned positive model;

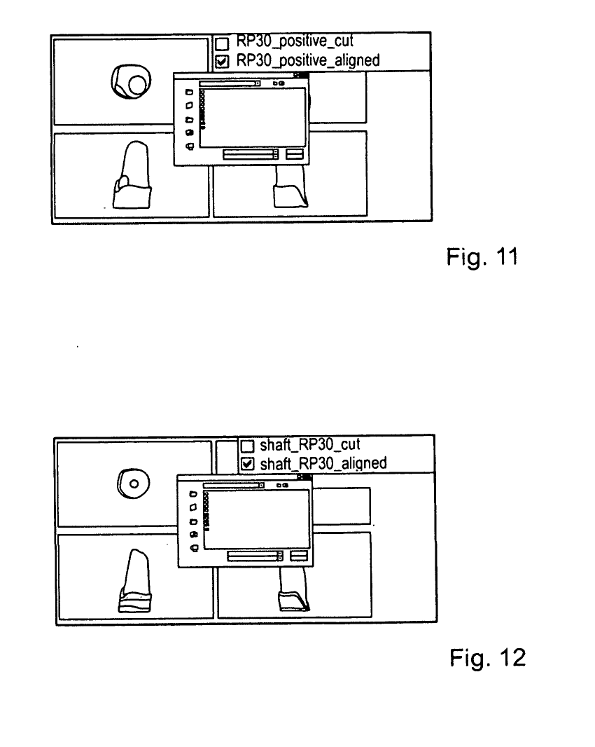

FIG. 12 the aligned shaft model;

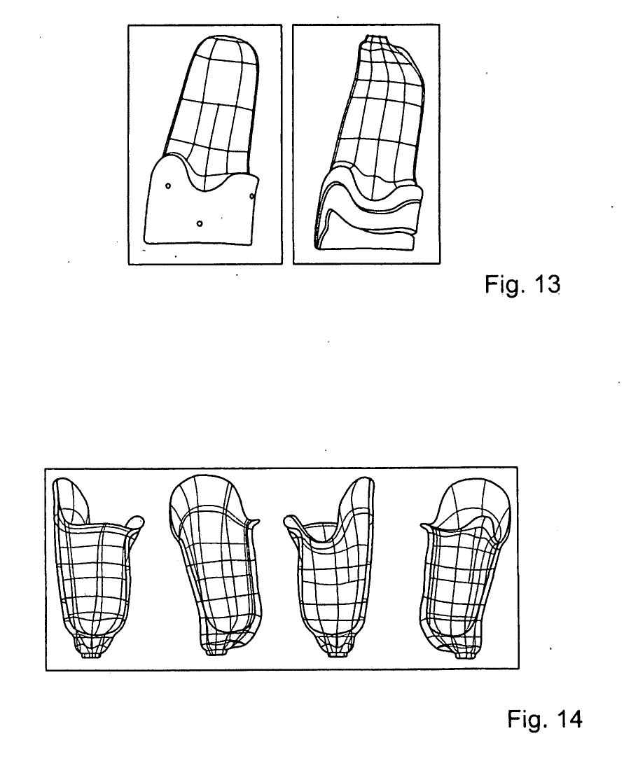

FIG. 13 the shaft model and the positive model after surface reconstruction;

FIG. 14 a view of the definite shaft after an import of the shaft model and of the positive model to a construction program;

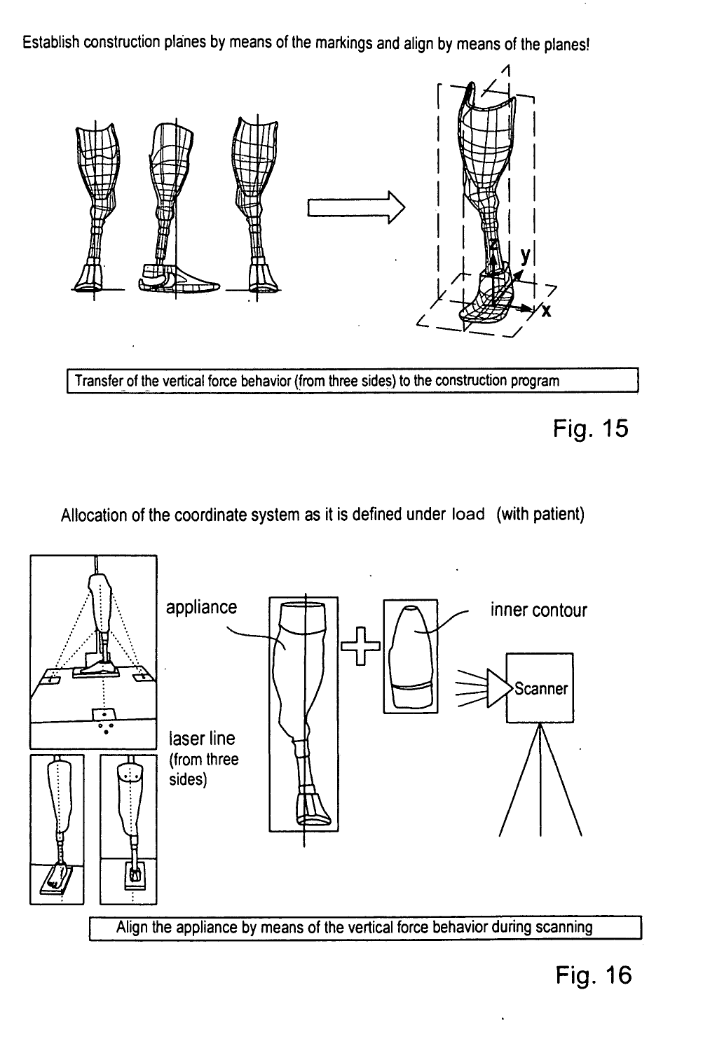

FIG. 15 a schematic diagram for illustrating the positioning of the shaft model pursuant to FIG. 14 in a load- or patient-oriented coordinate system;

FIG. 16 an alternative solution for aligning the shaft model in the patient/load-oriented coordinate system;

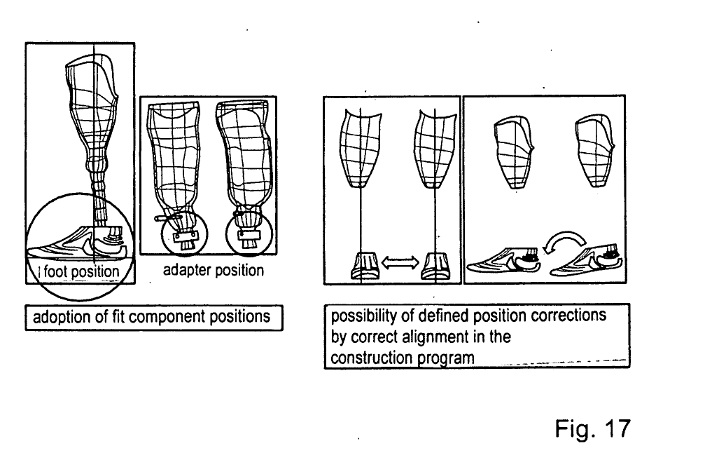

FIG. 17 the construction model pursuant to FIG. 14 with attached fit components;

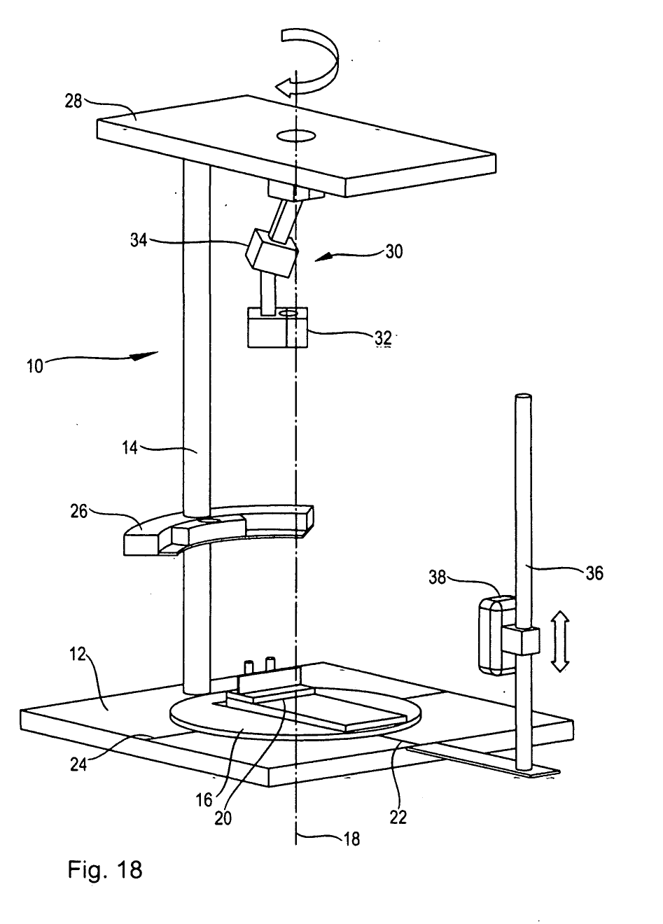

FIG. 18 an adjustment device for fixing the test appliance to be measured in position; and

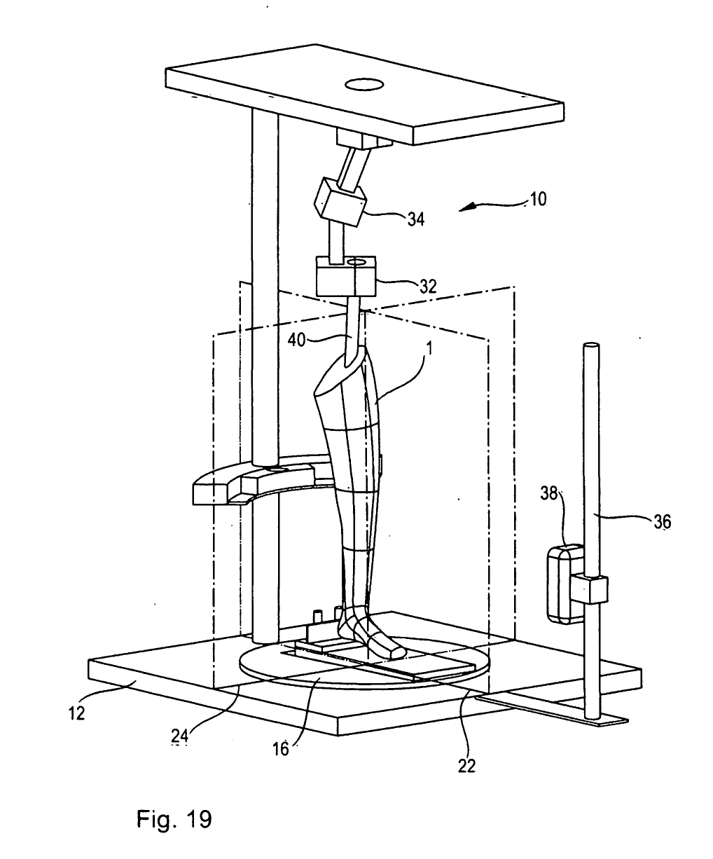

FIG. 19 the adjustment device pursuant to FIG. 18 with a clamped test appliance.

By means of FIG. 1 the basic proceeding during the transfer of statics from a test appliance or an appliance to be replaced to a “new” appliance (definite appliance) is explained.

In one variant of the invention, grouting of the test appliance (TA) is accordingly performed first of all, wherein care is taken during grouting that a protrusion or the like is formed which does not belong to the actual test appliance contour.

Then, auxiliary lines are assigned to this grouted test appliance. These auxiliary lines may, for instance, be indicated by means of projection or else be applied on the test appliance. As already explained, different auxiliary lines, for instance, verticals to the patient's footprint with load or without load, dotted function lines of floor reaction forces and of the force behavior with load on the appliance, or else construction reference lines or the like may be used so as to enable a positioning of the model in a patient- or load-oriented coordinate system in later method steps.

In a subsequent step, markings may be applied on the protrusion. This applying of markings may be renounced if the protrusion itself is designed as an auxiliary geometry/marking contour.

In a further step, the test appliance is then scanned along with the protrusion and/or the auxiliary geometry by means of a 3D scanner. Subsequently, the test appliance is removed, so that the positive representing the inner contour of the test appliance remains. This positive comprises the protrusion that was imaged during the scanning of the test appliance, so that a relative positioning of the models of the outer contour and of the inner contour may be performed in the following by means of this protrusion.

This relative position, however, positions the two models only relative to each other. By means of the initially explained auxiliary lines, the models are then aligned in the patient- or load-oriented coordinate system in a final step. As will be explained in detail in the following, it is possible to make this alignment by means of the auxiliary lines in the construction program for generating the construction model. The variant in which the alignment of the test appliance in the desired coordinate system is performed during scanning already, so that practically a largely automated alignment of the two models (outer contour/inner contour) is enabled in the construction program is somewhat more comfortable.

The variant of renouncing the grouting for producing the inner contour and of scanning the inner contour directly on the test appliance by means of a 3D laser is not illustrated in FIG. 1. The principal proceeding is, however, the same as in the afore-described embodiment.

It is pointed out that the term “protrusion” does not necessarily mean a proximal protrusion of the test appliance. This term comprises generally any geometric modification of the test appliance which can be scanned identically during the scanning of the inner and outer contours and thus facilitates the relative positioning of the models of the outer contour and of the inner contour.

In the following, a concrete proceeding for producing the models for a shaft and a positive according to the invention will be explained.

Pursuant to FIG. 2, the scanning of the grouted shaft is started with. Three markings are determined on the proximal protrusion and are not modified in the further proceeding. This proximal protrusion should not be modified in the following so as to maintain as many reference points as possible for a later congruent superimposing of the models obtained. As explained, the adapter position is also to be detected during scanning. Once scanning has been concluded, the model will be stored.

In the following step, the shaft is separated from the positive and the latter is scanned and is also stored (FIG. 3). This scan also shows the three markings.

Subsequently, both models available as point clouds are retrieved and cut digitally at the proximal protrusion. The markings applied are maintained in any case (FIG. 4).

In a following step the positive model is cut. For this purpose, the shaft is unmarked and masked. Cutting is then performed at the proximal protrusion with the markings being maintained (FIG. 5). Thus, the positive model of the protrusion is obtained.

Subsequently, the shaft model is processed in an appropriate manner. First of all, the positive model is unmarked and masked (FIG. 6), and cutting is performed at the proximal protrusion taking into account the markings, like with the positive model. This proximal protrusion is on principle identical with both models (FIG. 6). Both models should be cut in identical places if possible. Solid geometric auxiliary shapes would be conceivable which, however, have to be attached to the protrusion from the start.

In a following process step both cuts (shaft, positive) may then be superimposed in the program by means of the markings, wherein accuracy as high as possible has to be striven for (FIGS. 7, 8). Before the cutaway models thus obtained are stored in a suitable format (vvd), they should be renamed. Then, both cutaway models are stored individually. For this purpose, the respectively other cut is masked (FIGS. 9, 10). After the masking the complete model pertaining to the remaining cutaway model is opened (FIG. 11) and the cutaway model and the opened model are superimposed by means of the markings. The cutaway model, however, has to be the basis. Once this step has been concluded, the cutaway model is unmarked and the positive model is renamed, stored and exported in a suitable format (stl) (FIG. 11).

These process steps will then be repeated for the other model (shaft and shaft cut). This means that the shaft model and the pertaining shaft cut are opened and superimposed (by means of the markings). Also with this relative positioning the cutaway model has to form the basis. Once the models have been superimposed with an accuracy as high as possible, the cutaway model will be unmarked and masked. The shaft model will again be renamed, stored, and exported in the stl format (FIG. 12).

The aligned models are still available as point clouds. In a subsequent step, surface reconstruction of the positive model available as a point cloud is performed. Surface reconstruction of the shaft model available as a point cloud is not necessary since this outer contour merely is of subordinate significance for the fit and may be adapted to the respective conditions by the orthopedic specialist. It is to be understood that it is also possible to perform surface reconstruction for the shaft model. Care has to be taken that no new axial alignment of the models is performed (FIG. 13).

For the subsequent shaft modelling, both models that are positioned relative to one another are imported to the construction program, wherein care has to be taken during this import that—as explained before—the two models that are positioned relative to one another are arranged in the space, i.e. in a patient- or load-oriented coordinate system. This means that the models have to be positioned in the coordinate system such that the position during use is represented (see FIG. 14).

In this respect, a plurality of possibilities is generally available. Two of these possibilities will be explained by means of FIGS. 15 and 16.

The basis of both approaches is that the initially explained auxiliary lines (verticals oriented at the force behavior, oriented at floor reaction forces . . . ) are marked or projected. In the illustrated embodiment, three verticals to the footprint of the patient are illustrated, wherein theses verticals are assigned frontally, sagittally and dorsally with load. These auxiliary lines are imaged during scanning, so that the models of the outer and inner contours can be aligned in the construction program in the patient-oriented coordinate system prior to or after surface reconstruction by means of these auxiliary lines, so that the load case on the patient is exactly reproduced. These three planes that are positioned vertically on each other are indicated in FIG. 15.

In the alternative solution pursuant to FIG. 16, the test shaft is fixed prior to scanning in an adjustment device in the patient-/load-oriented coordinate system mentioned and is thus already positioned in the position of use. This means that a coordinate system as defined with load of the patient is already assigned during scanning. Then, the auxiliary lines mentioned are marked or projected. In the embodiment pursuant to FIG. 16, these auxiliary lines are projected by means of a laser in the three planes (frontal, sagittal, dorsal), so that they are also detected during scanning. After the scanning of the test shaft the latter is removed—the positive model remains in the adjustment device. After the scanning of the aligned positive model, both the shaft model and the positive model are available with the same axial alignment, so that, after the import to the construction program, both models are automatically aligned in the common patient-oriented coordinate system and are thus also positioned relatively.

The described proceeding enables the transfer of statics from the patient to the construction program, wherein the fit component positioning may also be assumed in the digital construction. The digital appliance construction will then be performed on principle in analogy to the approved handicraft method in the CAD program, wherein position corrections may be performed in a simple manner. Such corrections are illustrated in FIG. 17.

FIG. 18 illustrates an embodiment of an adjustment device 10 by means of which the test appliance 1 to be scanned (see FIG. 19) is held during the scanning process. This adjustment device 10 comprises a base plate 12 carrying a holding column 14. A rotary plate 16 rotatable about an axis of rotation 18 is mounted on the base plate 12. This rotary plate 16 carries a support 20 for the test appliance 1. In the base plate 12 a laser source for generating laser lines or planes 22, 24 is designed, so that auxiliary lines can be projected during the scanning process onto the test appliance 1 to be measured—as described in FIG. 16. The laser lines are arranged in frontal and sagittal position crosswise to one another. The planes projected practically via the laser light source are indicated in FIG. 19. A height-adjustable bracket 26 is formed at the holding column 14 for lateral support of the test appliance 1. The upper end of the holding column 14 holds a support plate on which a hinged fastener 30 is mounted to be rotated about the axis of rotation 18. This fastener 30 has a clamping element 32 by which the upper (view pursuant to FIGS. 16 and 19) end portion of the test appliance 1 is held. This clamping element 32 or retaining element is held on a hinged bracket 34 enabling an adjustment of the position of the clamping element 32 both in the vertical and in the horizontal directions, so that practically any desired holding position can be adjusted on the test appliance 1. It is understood that, for fixing the position, this hinged bracket 34 is designed with clamping elements fixing it in the desired relative position.

In accordance with the illustration in FIG. 18, a guidance 36 for a laser scanner (3D scanner) is further arranged on the base plate. This guidance 36 is designed such that the laser scanner 38 is adapted to be moved in vertical direction parallel to the axis of rotation 18. During scanning the test appliance 1 is rotated about the axis of rotation 18, so that the entire outer contour can be scanned by the laser scanner 38. It is understood that during this scanning process the laser scanner 38 can also be moved in the vertical direction. Both the rotation and the vertical movement of the laser scanner 38 may be performed by a motor.

As explained, the hinged bracket 34 comprises a plurality of degrees of freedom, so that it is possible to mount the object to be scanned without problems in the predetermined relative position with respect to the rotary plate 16.

FIG. 19 illustrates the adjustment device 10 according to FIG. 18 with a clamped test appliance 1. The three frontally and sagittally extending auxiliary planes via which the auxiliary lines mentioned are projected onto the test appliance are also illustrated. As already explained before, an adapter piece 40 is fastened in the test appliance 1 which is adapted to be brought into connection with the clamping element 32 for fixing so as to retain the test appliance in a patient-oriented position.

A device for performing the afore-described method variants thus comprises at least a 3D scanner for scanning the outer contour of the test appliance and the test appliance inner contour, a data storage for storing the data resulting from the scanning processes, and an evaluation unit via which surface resonstruction of the point cloud models available after scanning can be performed. Furthermore, means must be available by which the construction model can be calculated from these models obtained by surface resonstruction, wherein it is arranged in a patient-oriented coordinate system so as to be able to perform the afore-explained adaptation measures.

A largely automated production of the models is possible if the test appliance is positioned in the patient-oriented coordinate system via an adjustment device during scanning already, so that this positioning may be assumed automatically in the construction program.

Disclosed are a method for transferring statics of an orthopedic test appliance to a definite appliance and a device for performing this method. In accordance with the invention, the inner contour and the outer contour are scanned and aligned relative to each other by means of a protrusion/auxiliary geometry. In a further step, the models resulting from the scanning processes are positioned in a patient-/load-oriented coordinate system.

### LIST OF REFERENCE SIGNS

1 test appliance
10 adjustment device
12 base plate
14 holding column
16 rotary plate
18 axis of rotation
20 support
22 laser line
24 laser line
26 bracket
28 support plate
30 fastener
32 clamping element
34 hinged bracket
36 guidance
38 laser scanner
40 adapter piece

1. A method for transferring statics of an orthopedic test appliance to a defined appliance, comprising the steps of:
grouting the test appliance with a casting compound for forming a positive of the inner contour of the appliance such that a protrusion is formed, or attaching a protrusion to the test appliance,
wherein the protrusion is provided with markings or is formed with a predetermined marking contour, and/or
scanning the test appliance with adapters for applying fit components and with the protrusion;
separating the test appliance from the positive, and scanning the positive and the protrusion, or
scanning the inner contour of the test appliance;
storing the test appliance scan and the positive scan and/or the inner contour scan;
virtually superimposing the protrusions of the positive scan/inner contour scan and the test appliance scan by means of the markings and/or the marking contour;
unmarking the protrusions;
storing the appliance model such obtained; and
importing the appliance model to a construction program for generating the CAD data of a construction model for the definitive orthopedic appliance.

2. The method according to claim 1, wherein, after the superimposing of the protrusions, a point cloud test appliance model is available and is converted to the appliance model by surface reconstruction at least of the positive/inner contour point cloud model.

3. The method according to claim 1, wherein, prior to the virtual superimposing of the scans, the non-identical areas of the positive/inner contour point cloud and of the test appliance point cloud are masked, so that only cutaway models of the test appliance and of the positive or the inner contour remain.

4. The method according to claim 3, wherein, after the superimposing of the protrusions, the point clouds of the scans are made to overlap with the respective protrusion and the point clouds thus aligned are stored separately for the test appliance and the positive and/or the inner contour.

5. The method according to claim 1, wherein in the construction program both models are based on the same coordinate system.

6. The method according to claim 5, wherein auxiliary lines are assigned to the test appliance and/or the positive and/or the inner contour during scanning.

7. The method according to claim 6, wherein the test appliance and the positive model and/or the inner contour model are aligned in the construction program in the coordinate system by means of the auxiliary lines.

8. The method according to claim 6, wherein the alignment of the test appliance and/or of the positive is performed prior to scanning by means of the auxiliary lines.

9. The method according to claim 6, wherein the auxiliary lines are applied on the test appliance and/or on the positive with load in a front, side or rear view.

10. The method according to claim 1, wherein the appliance is an orthosis, a prosthesis, or a prosthesis shaft.

11. A device for performing the method according to claim 1, comprising:
a scanner for scanning the outer contour and the inner contour of the appliance, wherein a protrusion common to both scans is available;
a data storage for storing the scans;
an evaluation unit adapted
to align the two models of the outer contour and the inner contour relative to each other by means of the protrusions, and
means for generating CAD data for producing a construction model from the models aligned relative to each other and in a patient-oriented coordinate system.

12. The device according to claim 11, comprising an adjustment device for the defined positioning of the models of the inner contour and the outer contour of the test appliance in a particular coordinate system taking into account load conditions.

13. The device according to claim 11, wherein the evaluation unit is adapted to mask the areas of the point cloud models which do not belong to the protrusion, so that the relative alignment is performed on the basis of the remaining cutaway models.

14. The device according to claim 11, comprising at least one light source for the imaging of auxiliary lines on the test appliance outer contour and/or inner contour during scanning.

15. The device according to claim 14, wherein the auxiliary lines are verticals to a patient's footprint with load or without load, dotted function lines of floor reaction forces or of the force behavior with load on the appliance, or construction reference lines.

16. The device according to claim 11, wherein the adjustment device has a rotary plate as a support for the test appliance and a rotatably mounted fastener for an end section of the test appliance which is remote from the rotary plate.
